(19) United States
(12) Patent Application Publication
(10) Pub. No.: US 2012/0178181 A1
(43) Pub. Date: Jul. 12, 2012

(54) DEVICE AND METHOD FOR LABEL-FREE DETECTION OF DNA HYBRIDIZATION

(75) Inventors: Aoune Barhoumi, Houston, TX (US); Dongmao Zhang, Houston, TX (US); Nancy J. Halas, Houston, TX (US)

(73) Assignee: William March Rice University, Houston, TX (US)

(21) Appl. No.: 12/857,094

(22) Filed: Aug. 16, 2010

(51) Int. Cl. G01N 21/65; C07H 21/04; B82Y 15/00; B82Y 40/00

(52) U.S. Cl. 436/501; 536/24.3; 977/902; 977/896

(57) ABSTRACT

A device and method for detecting the hybridization of an unmodified target deoxyribonucleic acid (DNA) molecule including exposing a Raman substrate to the unmodified target DNA molecule, where the unmodified target DNA molecule is a complementary DNA molecule to a thiol-terminated probe DNA molecule covalently linked to the Raman substrate. Also, the thiol-terminated probe DNA molecule includes an adenine analog substituted for adenine. The hybridization of the unmodified target DNA molecule to the thiol-terminated probe DNA molecule is detected by measuring a Raman spectroscopic response of the Raman substrate.

### STATEMENT REGARDING FEDERALLY SPONSORED RESEARCH OR DEVELOPMENT

The present invention was made with government support under Contract Number F33615-03-D-5408 awarded by the Department of the Air Force. The government has certain rights in the invention.

### BACKGROUND

Detection of deoxyribonucleic acid (DNA) is critically important for many biological, clinical, forensic, security applications. Currently, the most common DNA detection methods are fluorescence-based, which may require expensive fluorescence dyes. In addition, fluorescence labeling techniques may be labor intensive and require a technologically intensive labeling process. Also, the quantification accuracy of fluorescence based methods may be poor due to the susceptibility of the fluorescence labels to photo-bleaching and spectral interferences from fluorescent impurities.

DNA hybridization has become one of the most frequent applied techniques for clinical laboratory screening of genetic and infectious diseases, as well as for forensic testing. In a typical DNA hybridization design, a probe DNA may be labeled with a radioactive or optical label for detection. As mentioned above, the most common DNA array techniques employ molecular fluorescent labels.

However, progress has been made in the development of alternative DNA tagging techniques such as gold nanoparticles, dye-doped silica nanoparticles, and quantum-dots as optical tags combined with various modalities of optical detection schemes. Although these approaches may have the potential to improve the detection limits in some techniques, they too, involve costly tagging chemicals and detection instrumentation.

Label-free detection has been emerging as a potential method for detecting DNA hybridization at a high sensitivity with low cost and low preparation time. Several formats for label-free detection have been proposed such as electronic, colorimetric, and electrochemical.

The hybridization of DNA when the DNA is attached to a surface has been studied for different situations, for example: different surfaces, capture probe sequences, packing densities, and buffers. However, experimentally determining DNA hybridization is known to be a time consuming task. Determining the hybridization of DNA usually requires dye-labeling the target sequence and determining the number of hybridized DNA sequences after displacing the target DNA molecules and the covalently bound DNA molecules from surface. Since the fluorescence of the dye labels may be pH dependent, keeping the solutions used at the optimum buffer conditions (pH and salt concentration) for dye fluorescence may be tedious, difficult, and introduce experimental errors.

Surface enhanced Raman spectroscopy (SERS) may be a promising alternative to achieve the label-free detection of DNA. By surface enhancing the Raman response of the DNA molecules before, during, or after hybridization with, for example, nanoshells, the label-free detection of DNA may be realized.

Nanoshells are spherical core-shell nanoparticles consisting of a silica core and gold or silver shell. The plasmon resonance frequencies of nanoshells are controlled by the relative inner and outer radius of the metallic shell layer. As such, the plasmon resonance frequency of a nanoshell may be tuned to wavelengths through out the visible and infrared regions of the electromagnetic spectrum. By tuning the relative plasmon resonance frequency, nanoshells may be used as the foundation for a reproducible SERS substrate.

### SUMMARY

In general, in one aspect, the invention relates to a method for detecting the hybridization of an unmodified target deoxyribonucleic acid (DNA) molecule. The method includes exposing a Raman substrate to the unmodified target DNA molecule, where the unmodified target DNA molecule is a complementary DNA molecule to a thiol-terminated probe DNA molecule covalently linked to the Raman substrate. The thiol-terminated probe DNA molecule includes an adenine analog substituted for adenine. The hybridization of the unmodified target DNA molecule to the thiol-terminated probe DNA molecule is detected by measuring a Raman spectroscopic response of the Raman substrate

In general, in one aspect, the invention relates to a device for determining the hybridization of a target DNA molecule. The device includes a thiol-terminated probe DNA molecule covalently linked to a Raman substrate. The thiol-terminated probe DNA molecule includes an adenine analog substituted for adenine and the thiol-terminated probe DNA is complementary to the target DNA molecule.

In general, in one aspect, the invention relates to a method of manufacturing a DNA molecule hybridization detector. The method includes covalently linking a thiol-terminated probe DNA molecule to a Raman substrate and passivating the Raman substrate with a blocking molecule. An amount of a target DNA molecule, complementary to the thiol-terminated probe DNA molecule, is quantified using a Raman spectroscopic response of the Raman substrate.

Other aspects of the invention will be apparent from the following description and the appended claims.

### BRIEF DESCRIPTION OF DRAWINGS

FIG. 1 shows a flow chart of a method in accordance with one or more embodiments of the invention.

FIGS. 2A-2E show schematics of the construction and use of a Raman based device for detection of DNA hybridization in accordance with one or more embodiments of the invention.

FIG. 3 shows a chart of the exemplary DNA molecules used in accordance with one or more embodiments of the invention.

FIG. 4 shows a chart of the Raman spectroscopic response of thermally pre-treated DNA in accordance with one or more embodiments of the invention.

FIG. 5 shows a chart of the spectral correlation function values of the Raman spectroscopic response of thermally pre-treated DNA in accordance with one or more embodiments of the invention.

FIG. 6 shows a chart of the Raman spectroscopic response of thermally pre-treated DNA in accordance with one or more embodiments of the invention.

FIG. 7 shows a chart of the label-free detection of DNA hybridization in accordance with one or more embodiments of the invention.

FIG. 8 shows a chart of the label-free detection of DNA hybridization in accordance with one or more embodiments of the invention.

FIG. 9 shows a chart of the Raman spectroscopic response of adenine and an adenine analog in accordance with one or more embodiments of the invention.

FIG. 10 shows graph of a Raman spectroscopic response of a DNA sequence with an adenine analog in accordance with one or more embodiments of the invention.

FIG. 11 shows a chart of the label-free detection of DNA hybridization in accordance with one or more embodiments of the invention.

FIG. 12 shows a calibration curve of the hybridization efficiency in accordance with one or more embodiments of the invention.

### DETAILED DESCRIPTION

Specific embodiments of the invention will now be described in detail with reference to the accompanying figures. Like elements in the various figures are denoted by like reference numerals for consistency.

In the following detailed description of embodiments of the invention, numerous specific details are set forth in order to provide a more thorough understanding of the invention. However, it will be apparent to one of ordinary skill in the art that the invention may be practiced without these specific details. In other instances, well-known features have not been described in detail to avoid unnecessarily complicating the description.

In general, one or more embodiments of the invention relate to a method and device for detecting the hybridization of DNA. Specifically, one or more embodiments of the invention relate to a method and device for using Surfaced Enhanced Raman Spectroscopy (SERS) to detect the hybridization of an unmodified target DNA molecule.

One or more embodiments of the invention relate to a method for detecting or quantifying the hybridization of an unmodified target DNA molecule by measuring the Raman spectroscopic response of the adenine groups in the target DNA molecule.

One or more embodiments of the invention relate to an apparatus or device for determining the hybridization of a target DNA molecule by measuring the Raman spectroscopic response of the adenine groups in the target DNA molecule. One or more embodiments of the invention relate to a method for manufacturing a target DNA molecule hybridization detector.

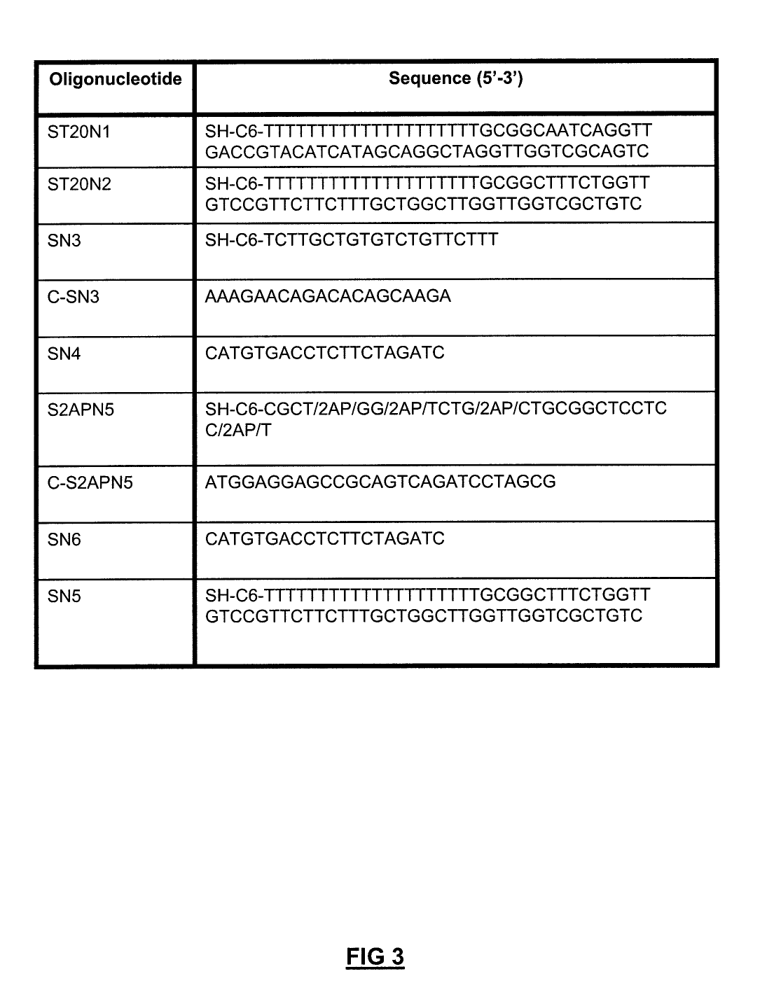

In one or more embodiments of the invention, a thiol-terminated DNA molecule refers to a molecule that includes a DNA sequence of amino acids which is terminated with a thiol group. The thiol-group is present to facilitate the bonding of the molecule to a substrate. The thiol-terminated DNA molecule may include an alkane spacer between the amino acid sequence and the thiol group. Examples of thiol-terminated DNA molecules used in one or more embodiments of the claimed invention are shown in FIG. 3.

One or more embodiments include acquiring a thiol-terminated DNA molecule, which may, for example, be purchased. Thiol-terminated DNA sequences may also be synthesized according to known techniques. In accordance with one or more embodiments of the claimed invention, the thiol-terminated DNA sequence may or may not include an adenine analog. That is, the thiol-terminated DNA molecules may have an adenine analog substituted for adenine in the DNA sequence.

In one or more embodiments of the invention, an adenine analog refers to any molecule that may be substituted for adenine in a DNA molecule. The adenine analog may be substituted for the adenine bases in a DNA molecule according to known techniques. The adenine analog is chosen such that the adenine substituted DNA molecule has a similar binding specificity and affinity as a DNA molecule with the adenine.

In one embodiment of the invention, a blocking molecule refers to a molecule designed to fill any gaps not occupied by the thiol-terminated DNA molecule. A blocking molecule may passivate the unused areas of the Raman substrate. Accordingly, the blocking molecule may hinder any non-specific binding of DNA molecules to the Raman substrate. Further, the blocking molecules may effect the orientation of the thiol-terminated DNA molecules on the surface. For example, a blocking molecule may influence the thiol-terminated DNA molecule to orientate perpendicular to the surface of the Raman substrate.

In one embodiment of the invention, a Raman substrate refers to substrate capable of enhancing the Raman spectroscopic response of a molecule when the molecule is in the vicinity of the surface. One of ordinary skill in the art will appreciate that embodiments of the present invention are not limited to any type of specific Raman substrate. The Raman substrate may only be limited by the ability of the thiol-terminated DNA molecules to covalently bind to the surface of the Raman substrate. However, as known by those of ordinary skill, the magnitude of the Raman enhancement may be determined by the type of Raman substrates. Examples of some Raman substrates include, but are not limited to, lithographic patterned metal surfaces, electro-chemical or vapor deposited metal surfaces, and colloidal arrays. In one embodiment of the invention, the Raman spectroscopic response corresponds to the measurement of Raman scattered light by a Raman instrument.

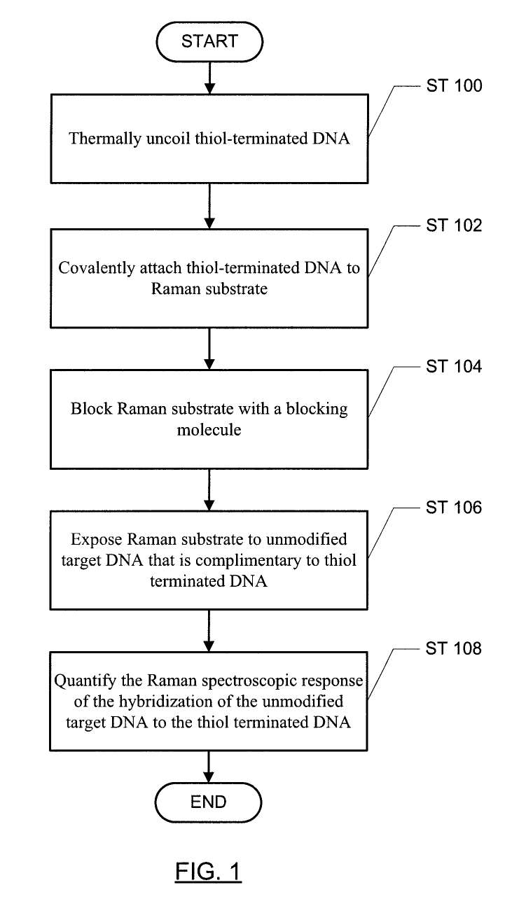

FIG. 1 shows flow chart outlining the methods in accordance with one or more embodiments of the invention. While the various steps in these flowcharts are presented and described sequentially, one of ordinary skill will appreciate that some or all of the steps may be performed in a different order, may be combined or omitted, and some or all of the steps may be performed in parallel.

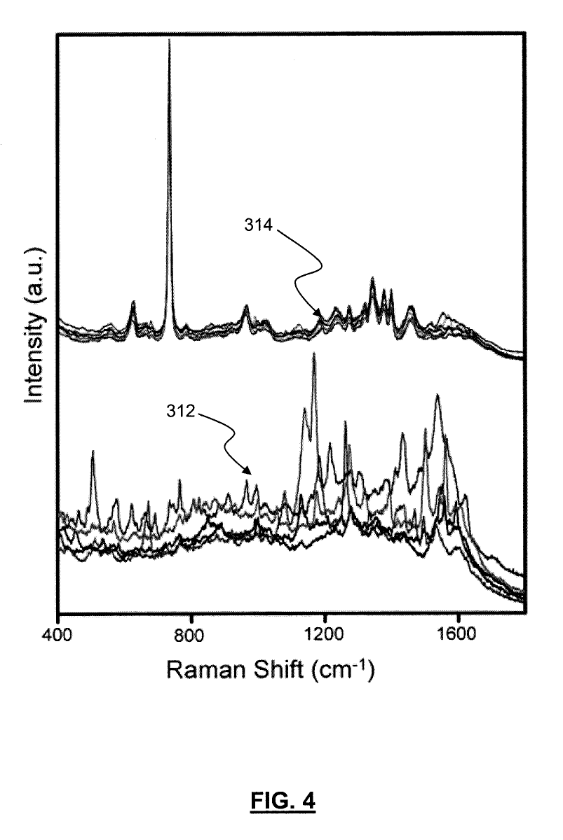

Referring to FIG. 1, in ST 100, the thiol-terminated DNA molecules are thermally uncoiled prior to attachment with a Raman substrate. In one or more embodiments of the invention, thermally uncoiling the thiol-terminated DNA molecules may be achieved by heating the thiol-terminated DNA molecules in solution followed by a rapid cooling. The thermal uncoiling of the DNA molecules is discussed further in relation to FIGS. 4-6.

In ST 102, the thiol-terminated DNA is covalently attached to a Raman substrate. Immobilization of a thiol-terminated DNA molecule may be accomplished by an incubation period of the thiol-terminated DNA molecule solution on a Raman substrate. For example, a solution of the thiol-terminated DNA molecule may be disposed on the Raman substrate overnight. Then, the Raman substrate may be rinsed to remove any excess thiol-terminated DNA molecules.

In ST 104, after the attachment of the thiol-terminated DNA molecule to the Raman substrate, the surface of the Raman substrate may be passivated with a blocking molecule. A blocking molecule may be used to occupy any areas of a surface not occupied by thiol-terminated DNA. For example, alkanethiols or a hydroxide terminated alkanethiol, such as mercaptonhexanol, may be used to passivate the Raman substrates. The blocking molecule chosen to passivate the Raman substrates may be chosen based on its size, binding affinity, or functional moieties.

At this stage, in accordance with one or more embodiments of the invention, the thiol-terminated DNA functionalized Raman substrate may be used as a device for the detection of an unmodified (or modified) complementary target DNA sequence. Also, the thiol-terminated DNA functionalized Raman substrate may also be used in a method for detecting the hybridization of an unmodified (or modified) complementary DNA sequence.

In ST 106, the thiol-terminated DNA molecule may be exposed to an unmodified (or modified) target DNA molecule. If the target DNA molecule has a DNA sequence complementary to the thiol-terminated DNA molecule, the target DNA molecule may then hybridize to the thiol-terminated DNA molecule. Hybridization may be carried out on the Raman substrate by adding the target DNA molecules in a solution of a proper hybridization buffer onto the thiol-terminated DNA molecules bound to the Raman substrate. Examples of proper hybridization buffers include Tris EDTA (TE) or Tris EDTA/NaCl buffers.

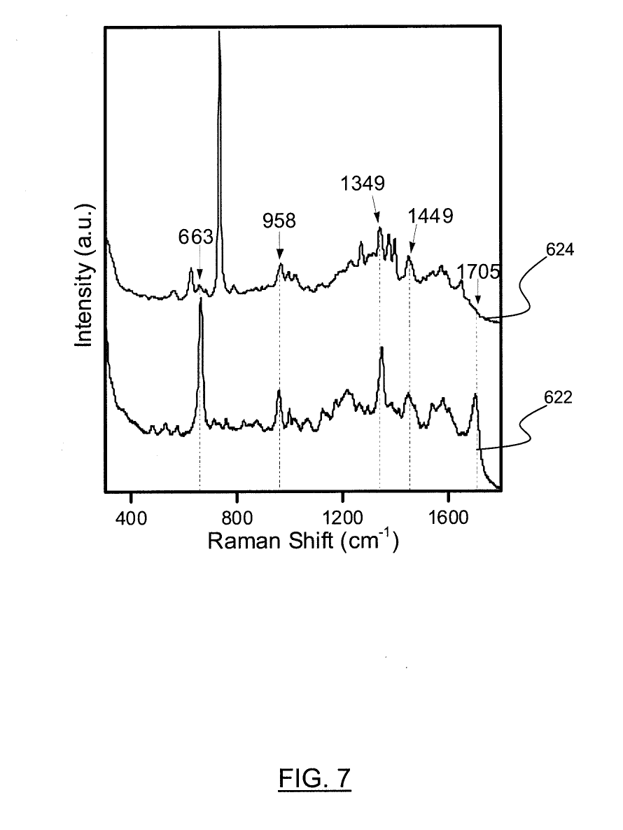

In ST 108, the Raman spectroscopic response before and/or after the hybridization of the target DNA molecule and thiol-terminated DNA molecule may be measured according to known techniques. The hybridization may be quantified using the Raman spectroscopic response of the different species involved. The Raman spectroscopic response of the hybridization of the target and thiol-terminated DNA molecules is discussed further in relation to FIGS. 7-8 and 11-12. Those skilled in the art will appreciate that the invention is not limited to the Raman spectroscopic response examples disclosed below.

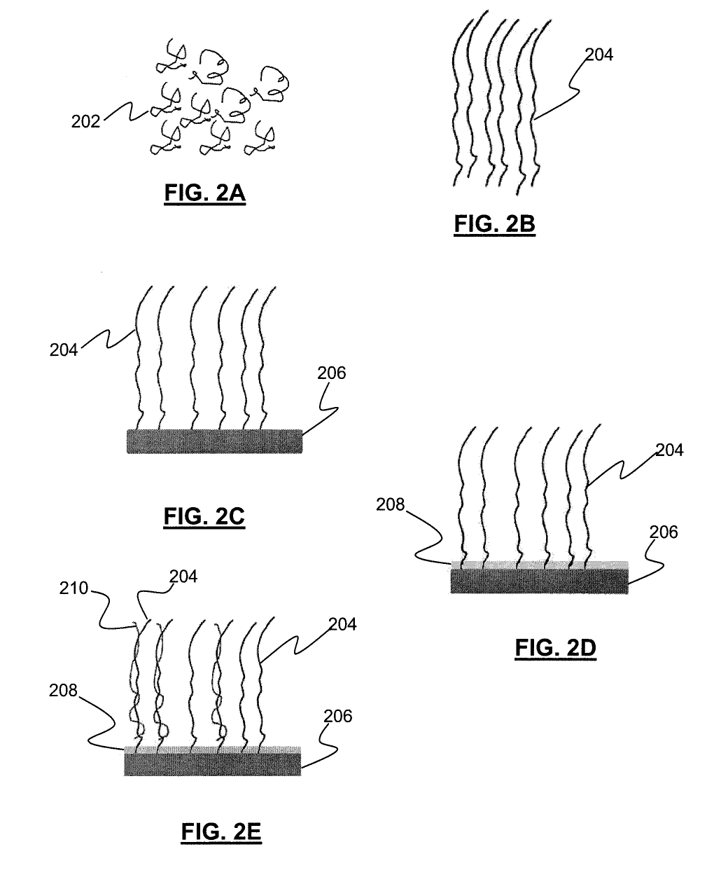

FIG. 2 shows a schematic of the methods of FIG. 1 in accordance with one or more embodiments of the invention. Referring to FIG. 2A, prior to use, the thiol-terminated and possibly adenine-substituted DNA molecules 102 may be reduced according to techniques known in the art. For example, the thiol-terminated and possibly adenine-substituted DNA molecules may be reduced with 1,4-Dithio-DL-threitol to break any disulfide moieties formed between different thiol-terminated DNA molecules. Samples may also be purified according to techniques known in the art. For example, NAP 5 purification columns or with other methods may be used to purify solutions of DNA molecules.

In accordance with one or more embodiments of the invention, to ensure high quality spectral acquisition, the thiol-terminated DNA molecules may be thermally uncoiled by heating the DNA solutions (ST 100 and FIG. 2B). For example, DNA molecules in a TE buffer (1×Tris EDTA buffer at a pH=7.5) may be heated to 95° C. for 10 to 15 minutes and then followed by rapid cooling in an ice bath. The uncoiled thiol-terminated DNA molecules 104 may then be covalently attached to a Raman substrate 106, via the thiol group (ST 102 and FIG. 2C). Immobilization of the thiol-terminated DNA molecules may be accomplished by overnight incubation of thiol-terminated DNA molecule 104 solution on the SERS active Raman substrate 106. Any excess thiol-terminated DNA molecules may then be removed by rinsing a buffer. The thermal uncoiling of the DNA molecules is discussed further in relation to FIGS. 4-6.

As stated previously, one of ordinary skill in the art will appreciate that embodiments of the present invention are not limited to any type of specific Raman substrate. One or more embodiments of the claimed invention may utilize metal nanoshells deposited on a surface as a Raman substrate. Metal nanoshells may be manufactured according to U.S. Pat. No. 6,344,272 hereby incorporated by reference in its entirety. For example, a metal nanoshell including a silica core with a gold shell was used for measuring the Raman spectroscopic response for one or more embodiments of the invention. The dimensions of the silica core and the gold shell were adjusted according to know techniques such that the peak plasmon resonance in an aqueous suspension was ˜785 nm. The 785 nm peak plasmon resonance was chosen to correspond to the excitation wavelength of the micro-Raman system used according to known techniques.

The nanoshell-based Raman substrates may include dispersed nanoshells bound to glass or quartz substrates. For example, a piranha cleaned fused quartz substrate may be incubated overnight in an (1%) ethanolic solution of poly(4-vinylpyridine) with a MW=160,000 and then dried with nitrogen gas. Subsequently, a volume of the aqueous nanoshell solution, for example 100 μl, may be deposited onto the functionalized fused quartz substrate. The substrate may then be allowed to sit at room temperature for 3 to 4 hours and then rinsed with Milli-Q water to remove any excess nanoshells. The fused quartz nanoshell functionalized substrates may then be dried with a gentle flow of nitrogen.

In one or more embodiments of the invention, to bind the thiol-terminated DNA molecules to the nanoshell based Raman substrates, an amount of thiol-terminated DNA molecules, 40-50 μL for example, of the thiol-terminated DNA molecule may be deposited onto a freshly made nanoshell SERS substrate. After some incubation time, overnight for example, the excess ssDNA (single stranded DNA) or dsDNA (double stranded DNA) solution may be removed by rinsing with TE or TE/50 mM NaCl buffer, respectively.

Referring now to FIG. 2D, to eliminate any possible nonspecifically bounded thiol-terminated DNA molecules and possibly prevent any nonspecific binding of the target DNA onto the Raman substrate surface, a blocking molecule 108 may be used in accordance with one or more embodiments of the invention (ST 104). For example, alkanethiols or hydroxide terminated alkanethiols may be used to passivate the SERS substrates. The blocking molecule chosen to passivate the Raman substrates may be chosen based on its size, binding affinity, or functional moieties.

Referring now to FIG. 2E, DNA hybridization may be carried out in situ (on the Raman substrate 106) by adding the target DNA molecules 110 in a solution of a proper hybridization buffer (TE/50 mM NaCl, pH=7.5 for example) onto the thiol-terminated DNA molecules 104 bound to the Raman substrate 106 (ST 106). To facilitate hybridization, the solution may be thermally treated similar to the thermal uncoiling previously described. For example, the functionalized Raman substrate, while immersed in the target DNA, solution may be heated up to 95° C. and allowed to slowly cool down to room temperature. Any un-hybridized, excess DNA may be removed by rinsing with the hybridization buffer.

The hybridization of a thiol-terminated DNA molecules to the target DNA molecules may also be performed in solution and then bound to the surface of the Raman substrate in accordance with one or more embodiments of the invention. Hybridization in solution may be achieved by mixing two complementary DNA sequences, one of which is thiol-terminated, in a 1:1 molar ratio in DNA hybridization buffer, for example TE/50 mM NaCl at a pH=7.5, heating the solution to 95° C., and allowing the solution to cool slowly to room temperature in a large water bath. The dsDNA molecules may then be covalently bound to a Raman substrate by placing a volume, 50 μL for example, of the solution of the hybridized dsDNA molecules on the surface of the Raman substrate. After incubation, any excess DNA may be removed by rinsing with a buffer.

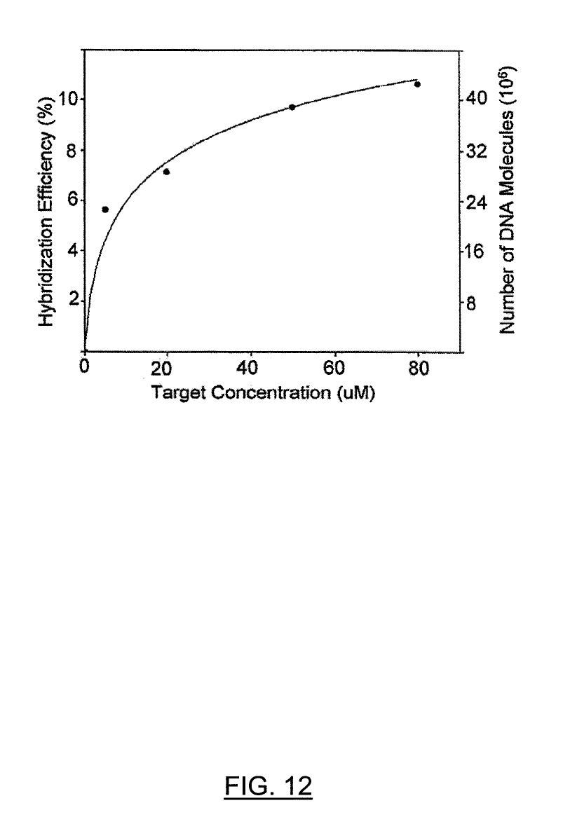

In accordance with one or more embodiments of the claimed invention, the gene expression level may be deduced from the hybridization efficiency calculated based on the measurement of the Raman spectroscopic response (ST 108). The details of the efficiency calculation and the gene level expression are discussed below with regard to FIG. 12.

One of ordinary skill in the art will appreciate that the measurement of the Raman spectroscopic response in accordance with one or more embodiments of the invention is not limited to any particular Raman instrument. For example, the Raman spectroscopic response may be measured using any Raman instrument known in the art. In one or more embodiments of the invention, the Raman spectroscopic response may be measured while substrates were immersed in an appropriate buffer, for example TE for ssDNA and TE/50 mM NaCl for dsDNA. The Raman instrument used to measure the Raman spectroscopic response may be a Renishaw in Via Raman microscope with 785 nm excitation wavelength. Backscattered light may be collected using a 63× water immersion lens, corresponding to a rectangular sampling area of 3 μm×30 μm. Unless stated otherwise, all the examples of SERS spectra disclosed herein were obtained with an integration time of 20 s and a laser power of 0.57 mW before the objective.

One of ordinary skill in the art will recognize that the conditions of the Raman spectroscopic response measurement are not limited to those stated above. For example, commercial Raman instruments are available that use excitation wavelengths at 514 nm, 532 nm, 632 nm, 785 nm, 1064 nm, etc. The excitation wavelength chosen may depend on the selection of the particular Raman substrate. One of ordinary skill in the art will recognize that the excitation intensity, sample area, collection time, and optical elements may all influence the measured Raman spectroscopic response according to know techniques.

FIGS. 4-12 further discuss aspects and specific examples of the device and methods of the claimed invention. The sequences of the DNA molecules used for exemplary purposes in accordance with one or more embodiments of the invention are shown in FIG. 3. The particular oligonucleotide sequences shown in FIG. 3 were obtained from Integrated DNA Technology Inc. One of ordinary skill in the art will appreciate that embodiments of the claimed invention are not limited to the DNA sequences disclosed in FIG. 3. One of ordinary skill in the art will also appreciate that the methods and device in accordance with one or more embodiments of the invention is not limited to DNA, but may be used for RNA (ribonucleic acid), or even DNA/RNA hybridization interactions.

Now referring to FIG. 4, SERS spectra of untreated (straight from the bottle) ssDNA 312 and thermally treated ssDNA 314 in accordance with one or more embodiments of the invention are shown. Multiple spectra are overlayed in both the untreated spectra 312 and the thermally treated spectra 314. The untreated spectra 312 in FIG. 4 clearly illustrate an extremely large variation in the SERS spectra that may be typically obtained prior to the thermal pretreatment of the DNA. Following the thermal treatment protocol, the SERS treated spectra 314 may appear to be dramatically different and may be highly reproducible. To evaluate the spectral reproducibility in the thermally cycled ssDNA 314, the average cross-correlation Γ between the individual spectra acquired was calculated for the untreated ssDNA spectra 312 and thermally treated ssDNA spectra 314 of the samples shown in FIG. 4.

One of ordinary skill in the art will appreciate that the thermal treatment may be repeated, or cycled, to achieve the desired results. Also, one of ordinary skill in the art will appreciate the thermal treatment is function of temperature and time. For example, a lower temperature for a longer period of time, or a higher temperature for a shorter period of time, may achieve the same results.

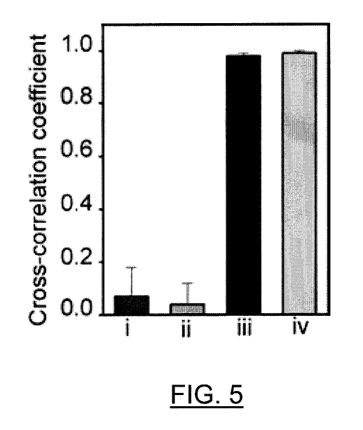

Referring now to FIG. 5, the average cross-correlation Γ between the untreated ssDNA spectra 312 and thermally cycled ssDNA spectra 314 in accordance with one or more embodiments of the invention is shown. The average cross-correlation Γ values clearly reflect the large variation in the SERS spectra between the untreated and thermally treated DNA molecules. It is clear that the thermally uncoiled ssDNA spectra 314 represented by column (iii) and pre-hybridized dsDNA represented by column (iv) have far higher average cross-correlation Γ values (Γ˜0.9) than the untreated samples (i) and (ii) (Γ˜0.1-0.2). It may be seen from the examples in FIG. 5 that the average cross-correlation Γ values obtained from the thermally cycled ssDNA spectra 314 represented by column (iii) and pre-hybridized dsDNA represented by column (iv) may provide a useful quantitative metric for assessing the SERS spectral reproducibility. Similar SERS spectra and spectral reproducibility has been observed for other DNA sequences, indicating that the observed increase in spectral reproducibility may be sequence independent.

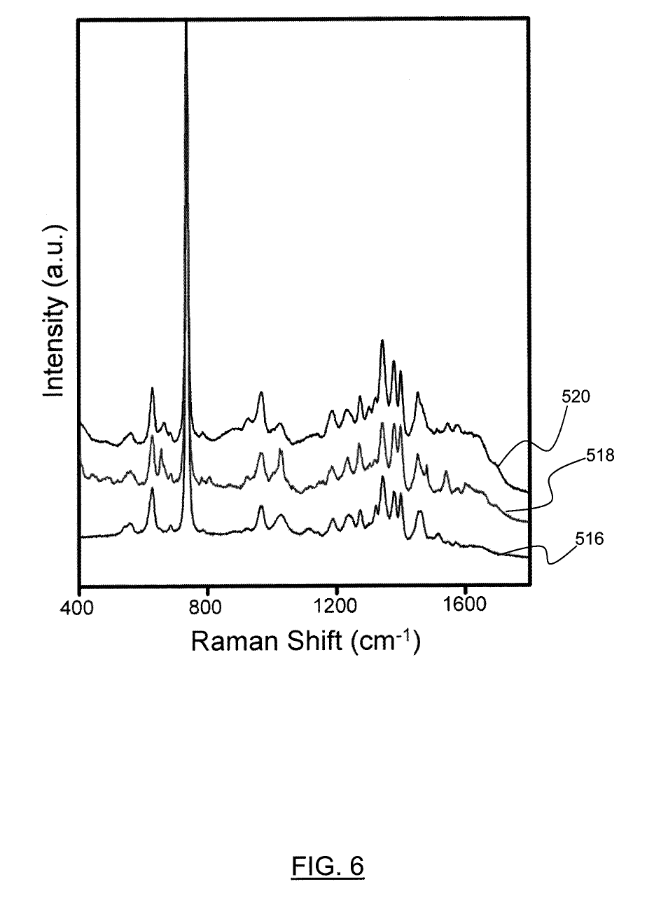

Referring now to FIG. 6, a direct comparison of the SERS spectra of adenine 516, a thermally pretreated, uncoiled 30 base ssDNA 518, and a thermally pretreated, pre-hybridized dsDNA (SN₅ and its complement) 520 in accordance with one or more embodiments of the invention are shown. Each spectrum shown in FIG. 6 is an average of 8 spectra collected from different locations of the same Raman substrate. The spectra shown in FIG. 6 are scaled and offset for clarity. It may be observed that the SERS spectra of ssDNA 518 and dsDNA 520 for this particular sequence, SN5, are dominated by the SERS spectrum of the adenine constituents. Under these experimental conditions, the Raman spectroscopic response from the adenine bases in the DNA oligomers may be more greatly enhanced than that of the other DNA bases. The only SERS spectral signature from the other DNA bases that may be observable in FIG. 6 is the weak 667 cm⁻¹ peak, which may be attributed to the ring breathing mode of guanine. The weak 667 cm⁻¹ peak appears in the SERS spectra of both the spectra of the ssDNA 518 and dsDNA 520, but is absent from the SERS spectrum of adenine 516. Raman spectral features from thymine and cytosine, and the backbone constituents of ribose and phosphate, may be indiscernible. The dominance of the adenine features may be observed in the SERS spectra for all thermally pretreated, uncoiled adenine-containing ssDNA, and pre-hybridized adenine-containing dsDNA samples tested.

Now referring to FIG. 7, the SERS spectrum of an adenine free DNA sequence 622, and a SERS spectrum of the same DNA sequence pre-hybridized with its complement sequence 624 in accordance with one or more embodiments of the invention is shown. The SERS spectra are averages of at least 8 spectra acquired from different locations of the same Raman substrate to ensure SERS spectral reproducibility. FIG. 7 demonstrates the feasibility of using the Raman spectroscopic response of adenine peaks as a marker for DNA hybridization by using an adenine-free thiol-terminated DNA molecule. FIG. 7 shows the Raman spectroscopic response of an adenine-free single strained thiol-terminated DNA molecule 622 and the same adenine-free single strained thiol-terminated DNA molecule after being hybridized to its complementary target DNA molecule 624. The Raman spectroscopic response of the thiol-terminated probe/target hybridized DNA molecule 624 appear to be dominated by the adenine bases in the complementary sequence of the unmodified target, making adenine an important marker of DNA hybridization.

In FIG. 7, the adenine-free thiol-terminated probe DNA molecule sequence SN5 is shown in FIG. 3. The dominance of the adenine Raman spectroscopic response may be primarily attributed to the high SERS cross section of adenine. The pre-hybridized DNA SERS spectrum 624 appears similar to an adenine SERS spectrum, dominated by the 736 cm⁻¹ peak (adenine breathing mode). In the presence of adenine, the Raman spectroscopic response from other DNA bases (guanine, thymine and cytosine) may be inconsequential. SERS of adenine-free DNA 622, a DNA sequence that does not include adenine bases (ST₂₀N2), may show the Raman spectroscopic response of other bases, particularly guanine. The main Raman peak of the adenine-free DNA spectrum 624 may be seen at 663 cm⁻¹, the guanine breathing mode.

FIG. 7 shows that the 736 cm⁻¹ mode of the Raman spectroscopic response of adenine is very distinctive for adenine bases. The adenine 736 cm⁻¹ SERS mode may be used to detect the presence of a modified or unmodified target DNA molecule that includes adenine and capable of hybridizing to a thiol-terminated DNA molecule, which is adenine free.

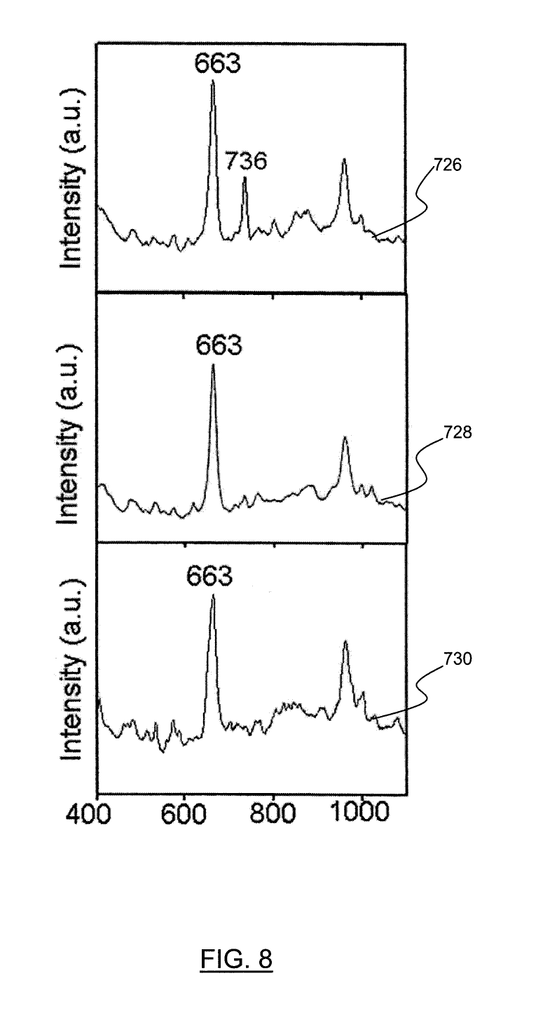

Now referring to FIG. 8, the Raman spectroscopic response of an unmodified target DNA molecule hybridization based on adenine-free thiol-terminated DNA molecule in accordance with embodiments disclosed here is shown. In FIG. 8, the Raman spectroscopic response of a thiol-terminated DNA molecule, covalently attached to a Raman substrate, is hybridized with a complementary unmodified target DNA molecule 726 is shown. Also in FIG. 8, the Raman spectroscopic response of a thiol-terminated DNA molecule, covalently attached to a Raman substrate, is hybridized with non-complementary unmodified target DNA molecule 728 is shown. FIG. 8 also includes the Raman spectroscopic response of the adenine-free thiol-terminated DNA 730.

In the adenine-free thiol-terminated DNA molecule example, the single stranded adenine-free DNA molecules were first immobilized on a gold nanoshell SERS active substrate through a thiol moiety on their 5′ end. As such, the rest of the DNA sequences are available for hybridizing with the complementary target molecules. The Raman spectroscopic response for the adenine-free thiol-terminated DNA molecule 730 is shown in FIG. 8 with the main Raman peak being at 663 cm⁻¹ (guanine breathing mode). A target sequence 726 (complementary to the adenine-free thiol-terminated DNA molecule) and a random non-complementary target DNA sequence 728 (non-complementary to the adenine-free thiol-terminated DNA molecule) were each separately hybridized with the adenine-free thiol-terminated DNA molecule. The hybridization of the complementary target DNA molecule 726 is evident by the appearance of the 736 cm⁻¹ adenine Raman response. For the non-complementary control sequence 728, a very small Raman spectroscopic response appeared at 736 cm⁻¹ which may be due to DNA/DNA interaction or DNA/surface non-specific binding with the Raman substrate.

A person of ordinary skill in the art will recognize, for on-surface DNA hybridization, the packing density of the bound DNA molecule may greatly affect the hybridization efficiency. Lower bound DNA molecule packing density may significantly increase the hybridization efficiency because the lower bound DNA molecule packing density may allow for a better interaction between the bound DNA molecule and the target DNA molecule. On the other hand, lower packing density on the Raman substrate of the bound DNA may provide more free space between bound DNA molecules for DNA molecule/surface non-specific binding. The non-specific binding between the DNA molecule and surface may be significant for a Raman substrate with a gold surface due to the high affinity between single stranded DNA and gold. To overcome the problem of non-specific binding associated with the DNA molecule/surface, mercaptohexanol may be used as a blocking molecule to passivate the free surface of the Raman substrate and prevent a target DNA molecule from interacting with the surface.

A person of ordinary skill would recognize that the present invention is not limited to mercaptohexanol as a blocking molecule. For example, alkanethiols or other hydroxide terminated alkanethiols may be used. Also, a person ordinary skill would recognize the influence that the concentration and deposition time of the blocking molecule may have on the thiol-terminated DNA molecule surface coverage. For example, a 7 nM solution of mercaptonhexanol with a 7 hour exposure may be sufficient to effectively passivate the Raman substrate. The concentration and exposure time of the blocking molecule is chosen to allow the passivating of the Raman substrate surface, while still maintaining sufficient coverage of the thiol-terminated DNA molecule to the Raman substrate.

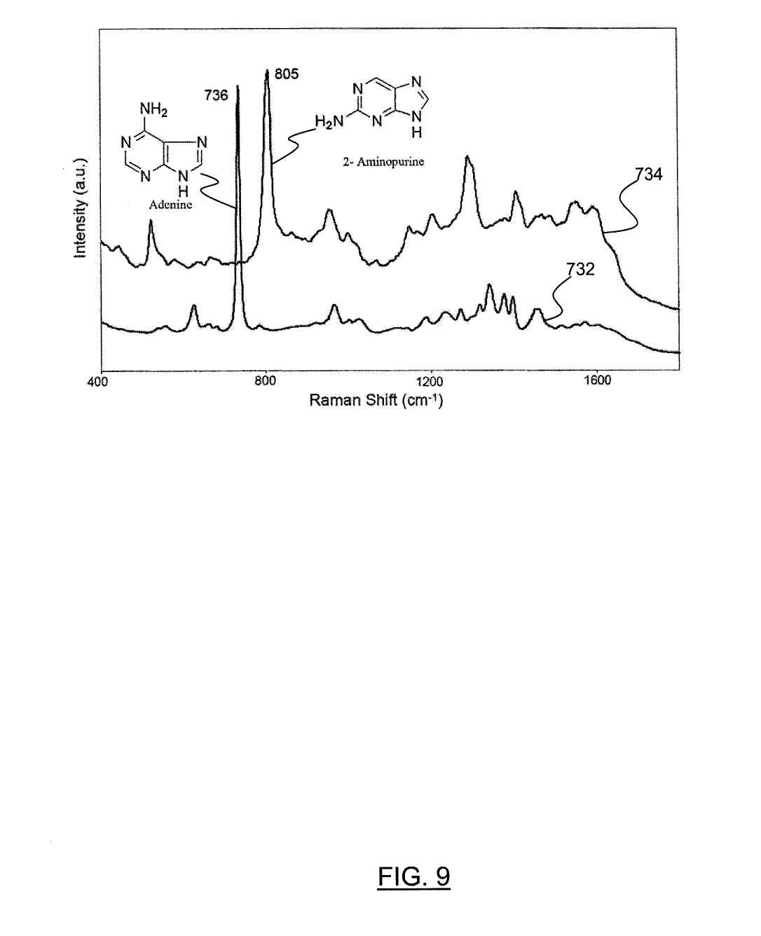

Referring now to FIG. 9, the Raman spectroscopic response of a DNA molecule including adenine and the Raman spectroscopic response of 2-aminopurine in accordance with one or more embodiments of the invention is shown. FIG. 9 shows the SERS spectra obtained with adenine 732 and 2-aminopurine (2-AP) 734, an adenine analog known to have similar binding characteristics as that of adenine. The spectra in FIG. 9 were taken under the same conditions. The similar spectral signal to noise ratio may indicate that the adenine 732 and 2-AP 734 have similar SERS activity, and the fact that there is no spectral overlapping in the major peaks in their respective Raman spectra demonstrate that 2-AP 734 may be an effective adenine analog for SERS detection of DNA hybridization. The quantification of the Raman spectroscopic response may be critical in quantitative analysis of the hybridization of DNA molecules.

One of ordinary skill will recognize that 2-aminopurine is not the only known adenine analog. For example, known adenine analogs include 2,6-diaminopurine, 3-nitropyrrole, and 5-nitroindole.

The use of an adenine analog may overcome the problem of the thiol-terminated DNA molecule bound to the Raman substrate being limited to DNA molecules that do not include adenine. 2-aminopurine is known to be used as an artificial adenine substitution. The substitution of adenine by the adenine isomer 2-aminopurine, may preserve the same characteristics of the non-substituted sequence. Very similar to adenine, 2-aminopurine is known to bind to thymine through hydrogen bonding. The substitution may only cause a small perturbation of the nucleic acid structure.

As shown in FIG. 9, the Raman spectroscopic response of the 2-aminopurine bases 734 is quite different than adenine bases. Most importantly, the Raman spectroscopic response of 2-aminopurine 734 does not have a Raman spectroscopic response in the 736 cm⁻¹ region, which means that it may be used as an adenine substitution.

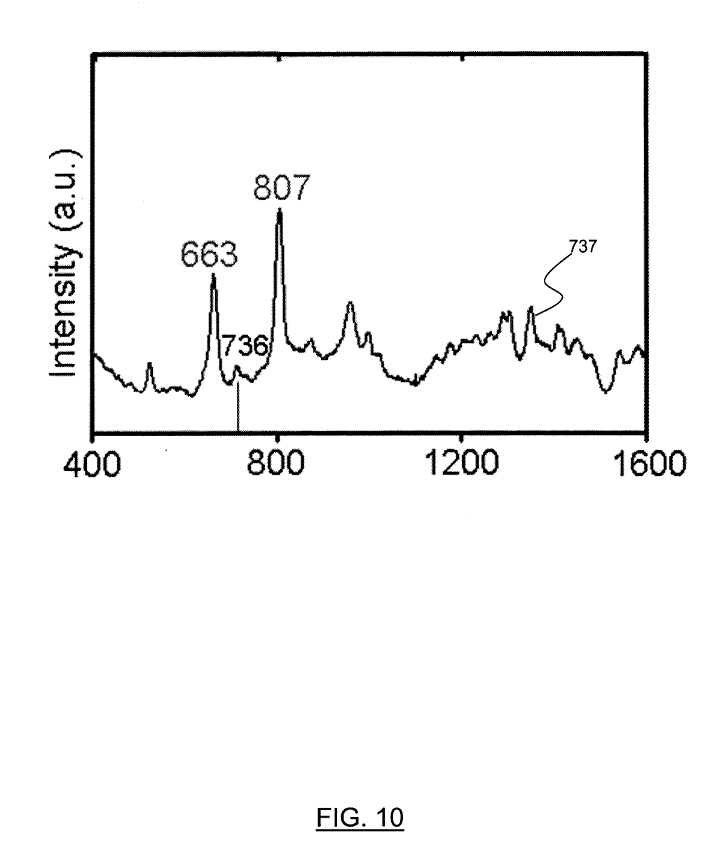

Referring now to FIG. 10, the Raman spectroscopic response of a DNA sequence containing 2-aminopurine in accordance with one or more embodiments of the invention is shown. The Raman spectroscopic response of a DNA sequence including 2-aminopurine 737, where the 2-aminopurine is substituted for adenine, shows only two Raman features at 807 cm⁻¹ (breathing mode of 2-aminopurine) and 663 cm⁻¹ (breathing mode of guanine). Therefore, a 2-aminopurine substituted thiol-terminated DNA molecule may be used as a label-free adenine-based detection system where the hybridization of the target DNA molecule may be indicated by the 736 cm⁻¹ adenine peak.

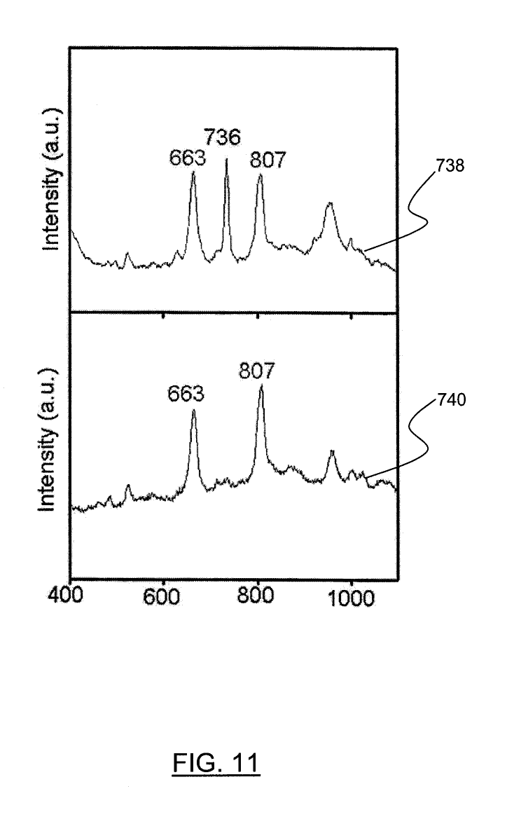

Referring now to FIG. 11, the label-free detection of DNA hybridization based on a 2-aminopurine modified thiol-terminated DNA molecule in accordance with one or more embodiments of the invention is shown. The Raman spectroscopic response of a complementary target DNA molecule hybridized to an adenine substituted thiol-terminated DNA molecule 738, substituted with 2-aminopurine, bound to a Raman substrate in accordance with one or embodiments is shown in FIG. 11. Also shown in FIG. 11 is a non-complementary target DNA molecule exposed to the 2-aminopurine adenine substituted thiol-terminated DNA molecule 740 shown as a control.

FIG. 11 shows the Raman spectroscopic response of the complementary target DNA molecule 738 and a non-complementary control 740. As can be seen in FIG. 11, the hybridization of the complementary target DNA sequence is identified by the 736 cm⁻¹ adenine peak.

Further spectral proof of the target DNA sequence hybridization, in addition to the 736 cm⁻¹ adenine peak, may be demonstrated by comparing the intensity of the ratio of the Raman spectroscopic response of the guanine peak at 663 cm⁻¹ to the 2-aminopurine peak at 807 cm⁻¹ between the complementary target 738 and the non-complementary target control 740 DNA molecule. A significant increase of the ratio of the Raman spectroscopic response of the guanine to the Raman spectroscopic response of the 2-aminopurine may be observed in the case of the complementary target hybridization. A relative increase in intensity of the guanine peak may indicate the hybridization of the complementary target sequence, which may contain guanine bases.

The hybridization of the complementary target sequence may be verified by the appearance of a new Raman peak at 736 cm⁻¹, the adenine Raman response, and/or a relative increase of the guanine peak.

In one or more embodiments of the invention, the SERS label-free detection method and device may provide a more straightforward way to determine hybridization efficiency. The DNA hybridization efficiency may be calculated based on the ratio of the 736 cm⁻¹ adenine peak intensity of the target DNA molecule to the 807 cm⁻¹ peak of the 2-aminopurine in the thiol-terminated DNA molecule. The intensity of the 807 cm⁻¹ 2-aminopurine peak may be constant and may be determined based on the thiol-terminated DNA molecule packing density. Therefore, the peak ratio is expected to be zero for non-hybridization. When the thiol-terminated DNA molecule and target DNA molecule are pre-hybridized prior to binding to the Raman substrate, the peak ratio is expected to be a maximum, corresponding to 100% hybridization efficiency. Different hybridization efficiencies may be extrapolated from the different peak ratios and correlated to the target DNA molecule concentration or gene level expression. The hybridization efficiency may be normalized for all Raman substrates because the aforementioned ratio of the Raman spectroscopic response only depends on the ratio of intensities of the Raman peaks.

One of ordinary skill in the art will appreciate, given the wealth of chemical structure information contained in the SERS spectra disclosed herein, that the above methods for determining the hybridization efficiency and target DNA molecule concentration are not limited to the ratios described above. For example, the presence of a peak at 736 cm⁻¹ is a direct measurement of the amount of adenine probed. The same is true for Raman spectroscopic response of guanine at 663 cm⁻¹. Further, other ratios of the adenine, guanine, and adenine analogs may be used to quantify the hybridization and molecular concentrations. Still further, the other Raman modes in the spectra disclosed herein are directly related to the chemical moieties and hybridizations involved and, as such, the invention is not limited to ratios or the Raman spectroscopic response disclosed above.

Referring now to FIG. 12, a calibration curve showing the hybridization efficiency versus target DNA molecule concentration in accordance with one or more embodiments of the invention is shown.

To determine the hybridization efficiency, the Raman spectroscopic response of pre-hybridized dsDNA may be obtained. When the thiol-terminated DNA sequence is hybridized to the target DNA sequence and then covalently attached to the Raman substrate, the ratio of the Raman spectroscopic response peak intensities between the adenine of the target DNA molecule and the adenine analog of the thiol-terminated molecule represent the case when 100% of the thiol-terminated/target DNA sequences are hybridized, it is thus denoted as R₁₀₀.

Then, in the case where the thiol-terminated DNA molecule is covalently bound to the Raman substrate followed by the hybridization of the target DNA molecule, the hybridization efficiency Eₓ may be calculated using the measured peak ratio Rₓ of the Raman spectroscopic response of the adenine in the target DNA molecules vs. the Raman spectroscopic response of the adenine analogs in the thiol-terminated DNA. The efficiency Eₓ may be calculated using Eₓ=100%×Rₓ/R₁₀₀. It should be noted that the R₁₀₀ measured as described above may be different for different thiol-terminated/target DNA pairs. The R₁₀₀ values may be obtained experimentally or empirically with help of theoretical modeling techniques known in the art.

FIG. 12 shows that the hybridization efficiency may be fairly low even at high target DNA molecule concentration. For example, as shown in FIG. 12, the hybridization efficiency is −11% when the target DNA molecule concentration is 80 μM. The low hybridization efficiency is consistent with known techniques and may be due to the thiol-terminated DNA molecule packing density and the hybridization conditions, such as the buffer used and temperature. The efficiency may also be influenced by the blocking agent used and the conditions of binding the blocking molecule to the substrate. One of ordinary skill in the art would recognize that using a spacer short DNA sequence may further improve the hybridization efficiency.

To determine any detection limits of the SERS label-free detection in accordance with one or more embodiments of the invention, the Raman spectroscopic response of the hybridization with decreasing target DNA molecule concentrations may be measured. For example, as shown in FIG. 12, the minimum target DNA concentration that may be detected and discriminated versus a control DNA sequence based on the Raman spectroscopic response peak ratios is ˜80 nM. As such, the minimum target DNA concentration may correspond to only 1.2×10⁶ detected target DNA molecules. 1.2×10⁶ target DNA molecules may correspond to the number of molecules on the 30 μm×3 μm sampling area of the Raman substrate and may be influenced by the thiol-terminated DNA molecule surface coverage. The number of molecules detected may be determined based on the surface coverage of previous reports from similar molecules and DNA hybridization efficiency. In this example, for 80 nM target concentration, the hybridization efficiency may be only 0.3%. Such a hybridization efficiency may be improved by developing the appropriate hybridization buffer and/or altering the thiol-terminated DNA molecule packing density. The detection limit for one or more embodiments described herein may not be determined by the target DNA concentration, but rather by the hybridization efficiency. By increasing the hybridization efficiency to, for example, 30%, the detection limit may be decreased to the femto-molar range.

One or more embodiments of the invention may provide a straightforward approach to study DNA hybridization efficiency for different DNA sequences, buffer conditions, spacers and so on. One or more embodiments of the invention may improve other DNA detection techniques as well as on on-surface DNA hybridization technologies.

Further, those of ordinary skill appreciate that DNA mutations may decrease the hybridization efficiency. Accordingly, one or more embodiments of the present invention may be used to detect DNA mutations such as, for example, SNP (single nucleotide polymorphism). A mutation on the target DNA molecule may decrease the hybridization efficiency, which in one or more embodiments of the present invention may be determined as a decrease in the peak ratio. Moreover, chemically modified DNA, for example, oxidized or methylated, may also have a lower hybridization efficiency which may be detected by one or more embodiments of the invention.

Particularly, DNA oxidation, which may occur most readily at guanine, and may be correlated to aging-related diseases, such as cancer, may be detected. The Raman spectroscopic response of oxidized guanine may be different than normal guanine. The presence of oxidized guanine on the target DNA sequence may be indicated by decrease in the ratio of the Raman spectroscopic response of the adenine of the target DNA molecule to Raman spectroscopic response of the 2-aminopurine in the thiol-terminated DNA molecule ratio due to lower hybridization efficiency associated with sequence perturbation. The presence of oxidized guanine on the target DNA sequence may also be indicated by a decrease in the ratios of the Raman spectroscopic response of guanine to the Raman spectroscopic response of the 2-aminopurine peak ratio because oxidized guanine may not have the same SERS features as native guanine. Also, the presence of oxidized guanine on the target DNA sequence has the possibility of the appearance of new SERS features associated with the oxidized guanine.

Further, the SERS spectra generated using SERS for DNA detection may be analyzed to obtain other useful information. Whereas other DNA detection techniques are based on detecting tags, one or more embodiments of the invention may be used as a detection scheme based on the direct detection of the Raman spectroscopic response of the DNA. As a result, the slightest variation on the target DNA sequence base composition and/or chemical structure may be easily detected. Embodiments of the claimed invention may be extended beyond simple DNA detection, to detecting mutated and chemically modified target DNA, which has the potential to be used in many biomedical applications. As such, one or more embodiments of the invention may be relevant to all biomedical applications involving target DNA detection.

One or more embodiments of the claimed invention, may allow the measurement of the target DNA molecule concentration based on a calibration curve, such as shown in FIG. 12. Whereas in other DNA detection techniques, target concentration is typically determined through a comparative study, in one or more embodiments of the invention, target DNA sequence concentration may be directly extrapolated from a calibration curve. The intensity ratio of the Raman spectroscopic response of the target adenine to the Raman spectroscopic response of the 2-aminopurine in the thiol-terminated DNA molecule may be directly correlated to the target concentration. As has been indicated by one or more embodiments of the invention, the thermal treatment of DNA may provide a high substrate to substrate SERS spectral reproducibility in terms of peak position, but not necessarily in Raman peak intensity.

In SERS, peak intensity may depend not only on the number, conformation, and relative proximity of molecules to the surface, but peak intensity may also depend greatly on substrate quality. To compare the Raman intensities of spectra acquired on different substrates may require high substrate reproducibility, which may be experimentally hard to achieve. One or more embodiments of the claimed invention may allow substrate to substrate comparison because the detection may be based on the peak ratios, regardless of the absolute intensity. For example, the peak ratio may only depend on the number of target DNA molecules with respect to the number of thiol-terminated DNA molecules, which represents the hybridization efficiency.

As stated previously, the packing density of the DNA molecules may be related to the number of hybridized DNA molecules through the hybridization efficiency. One or more embodiments of the invention may be able to determine the absolute number of target DNA molecules hybridized to the thiol-terminated DNA molecules covalently attached to the Raman substrate.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

What is claimed is:

1. A method for detecting the hybridization of an unmodified target deoxyribonucleic acid (DNA) molecule comprising:
exposing a Raman substrate to the unmodified target DNA molecule,
wherein the unmodified target DNA molecule is a complementary DNA molecule to a thiol-terminated probe DNA molecule covalently linked to the Raman substrate,
wherein the thiol-terminated probe DNA molecule comprises an adenine analog substituted for adenine; and
detecting the hybridization of the unmodified target DNA molecule to the thiol-terminated probe DNA molecule by measuring a Raman spectroscopic response of the Raman substrate.

2. The method of claim 1, further comprising:
quantifying a hybridization amount of the unmodified target DNA molecule hybridized to the thiol-terminated probe DNA molecule using an intensity of the Raman spectroscopic response of adenine from the unmodified target DNA molecule hybridized to the thiol-terminated probe DNA molecule.

3. The method of claim 2, wherein the hybridization amount is quantified by a ratio of the Raman spectroscopic response of the adenine of the unmodified target DNA molecule to the Raman spectroscopic response of the thiol-terminated probe DNA molecule.

4. The method of claim 1, wherein the adenine analog is one selected from a group consisting of 2-aminopurine, 2,6-diaminopurine, 3-Nitropyrrole, and 5-nitroindole.

5. The method of claim 1, wherein the Raman spectroscopic response is enhanced by the Raman substrate.

6. The method of claim 5, wherein the Raman substrate comprises metal nanoshells.

7. The method of claim 1, wherein the Raman spectroscopic response is measured using a laser in the wavelength range between 500 nm and 1100 nm.

8. A device for determining the hybridization of a target DNA molecule, the device comprising:
a thiol-terminated probe DNA molecule covalently linked to a Raman substrate,
wherein the thiol-terminated probe DNA molecule comprises an adenine analog substituted for adenine, and
wherein the target DNA molecule is complementary to the thiol-terminated probe DNA.

9. The device of claim 8, wherein
the thiol-terminated probe DNA molecule is thermally uncoiled prior to covalently linking the thiol-terminated probe DNA molecule to the Raman substrate; and
the Raman substrate is passivated with a blocking molecule.

10. The device of claim 9, wherein the blocking molecule is one selected from a group consisting of an alkanethiol or hydroxide terminated alkanethiol.

11. The device of claim 8, wherein an amount of the target DNA molecule is quantified using an intensity of the Raman spectroscopic response of adenine from the target DNA molecule.

12. The device of claim 11, wherein an amount of target DNA molecule hybridized to the thiol-terminated probe DNA molecule is quantified by a ratio of the Raman spectroscopic response of the adenine of the unmodified target DNA molecule to the Raman spectroscopic response of the thiol-terminated probe DNA molecule.

13. The device of claim 8, wherein the adenine analog is one selected from a group consisting of 2-aminopurine, 2,6-diaminopurine, 3-Nitropyrrole, and 5-nitroindole.

14. The device of claim 8, wherein the Raman spectroscopic response is enhanced by the Raman substrate.

15. The device of claim 8, wherein the Raman substrate comprises metal nanoshells.

16. The device of claim 8, wherein the Raman spectroscopic response is measured using a Raman instrument equipped with a laser in the wavelength range between 500 nm and 1100 nm.

17. A method of manufacturing a DNA molecule hybridization detector, the method comprising:
covalently linking a thiol-terminated probe DNA molecule to a Raman substrate; and
passivating the Raman substrate with a blocking molecule;
wherein an amount of a target DNA molecule, complementary to the thiol-terminated probe DNA molecule, is quantified using a Raman spectroscopic response of the Raman substrate.

18. The method of claim 17, further comprising:
thermally uncoiling the thiol-terminated probe DNA molecule prior to covalently linking the thiol-terminated probe DNA molecule to the Raman substrate.

19. The method of claim 17, wherein an amount of target DNA molecules hybridized to the thiol-terminated probe DNA molecule is quantified by a ratio of the Raman spectroscopic response of the target DNA to the Raman spectroscopic response of the thiol-terminated probe DNA molecule.

20. The method of claim 17, wherein
the Raman substrate comprises metal nanoshells which enhance the Raman spectroscopic response.
